(19) United States
(12) Patent Application Publication
(10) Pub. No.: US 2023/0147605 A1
(43) Pub. Date: May 11, 2023

(54) METHOD, DEVICE, AND SYSTEM FOR BLOOD OXYGEN SATURATION AND VITAL SIGN MEASUREMENTS USING A WEARABLE BIOSENSOR

(71) Applicant: Vital Connect, Inc., San Jose, CA (US)

(72) Inventors: Nersi Nazari, San Jose, CA (US); Olivier Colliou, San Jose, CA (US); Wenkang Qi, San Jose, CA (US)

(21) Appl. No.: 17/520,311

(22) Filed: Nov. 5, 2021

(51) Int. Cl. A61B 5/145; A61B 5/00

(52) U.S. Cl. CPC A61B 5/14542; A61B 5/002; A61B 5/0004; A61B 5/6824; A61B 5/6826; A61B 5/6898; A61B 2562/0219

(57) ABSTRACT

An unobtrusive ambulatory wearable biosensor device, system, and method continuously monitors blood oxygen saturation of a patient in their free-living conditions. The wearable biosensor system may include a finger sensor device, wrist module device, relay device, and wearable sensor patch.

### BACKGROUND

For many vital sign measurements such as blood oxygen saturation, a wearable biosensor is deployed that includes a sensor, a processor based system, and a screen to display the measurements. This arrangement is limited by the computational power required and display screen of the wearable biosensor to be comfortably worn by a user.

The present application overcomes the limitation of traditional wearable blood oxygen saturation monitors for continuous unobtrusive ambulatory monitoring of patient in their free-living conditions using a wearable biosensor. The present disclosure provides a solution to reduce or remove the computing processor and display for such a device while providing a better solution and at a lower cost.

### SUMMARY

In one example embodiment, a system to determine blood oxygen saturation (SpO2), includes: a finger sensor device including a sensor and a transmitter coupled to the sensor; a wrist module device communicatively connected to the finger sensor device via a cable; and a relay device communicatively connected to the wrist module device; wherein the sensor of the finger sensor device noninvasively measures changes of light absorption in oxygenated or deoxygenated blood as signal data and sends the signal data to the wrist module via the cable, wherein the wrist module receives the signal data and wirelessly sends the signal data to the relay device, wherein the relay device calculates a percentage of saturation of hemoglobin in the blood (Sp02) using the signal data, and wherein the relay device displays the calculated percentage of saturation of hemoglobin in the blood.

In another example embodiment, a method to determine blood oxygen saturation (SpO2), includes: noninvasively measuring, by a finger sensor device, changes of light absorption in oxygenated or deoxygenated blood as signal data; sending, by the finger sensor device, the signal data to a wrist module via a cable; wirelessly sending, by the wrist module device, the signal data to a relay device; calculating, by the relay device, a percentage of saturation of hemoglobin in the blood (Sp02) using the signal data; and displaying the Sp02 on a display of the relay device.

In yet another example embodiment, a biosensor device to determine blood oxygen saturation (SpO2), includes: a finger sensor coupled to a transmitter; and a wrist module communicatively connected to the finger sensor via a cable; wherein the finger sensor noninvasively measures changes of light absorption in oxygenated or deoxygenated blood as data and sends the data to the wrist module via the cable, wherein the wrist module receives the data and wirelessly sends the data to a relay device, wherein the relay device calculates a percentage of saturation of hemoglobin in the blood (Sp02) using the data, and wherein the relay device displays the calculated percentage of saturation of hemoglobin in the blood.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the brief description that follows, embodiments are described as illustrations only since various changes and modifications will become apparent to those skilled in the art from the following detailed description. The use of the same reference numbers in different figures indicates similar or identical items.

FIG. 1A shows an example illustration for implementing an embodiment of a system for measuring blood oxygen saturation.

FIG. 1B shows an example illustration for implementing another embodiment of a system for measuring blood oxygen saturation.

FIG. 2A shows an example illustration of a wireless wrist sensor device in accordance with an embodiment.

FIG. 2B shows an example illustration of a wireless fingertip sensor device in accordance with an embodiment.

FIG. 2C shows an example illustration of a wireless ring sensor device in accordance with an embodiment

FIG. 3A shows an example component block diagram of the wired sensor device of FIGS. 1A and 1B in accordance with an embodiment.

FIG. 3B shows an example component block diagram of the wired wrist module device of FIGS. 1A and 1B in accordance with an embodiment.

FIG. 3C shows an example component block diagram of the wireless sensor devices of FIGS. 2A - 2C in accordance with an embodiment.

FIG. 3D shows an example component block diagram of the relay device of FIGS. 1A and 1B in accordance with an embodiment.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

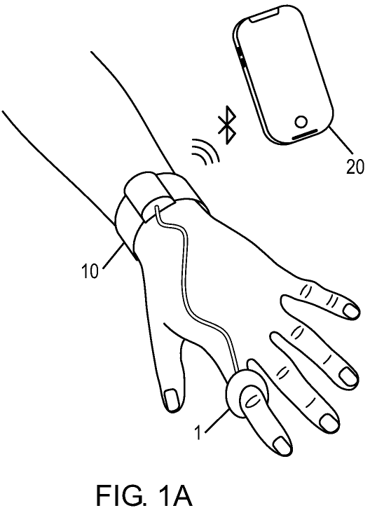

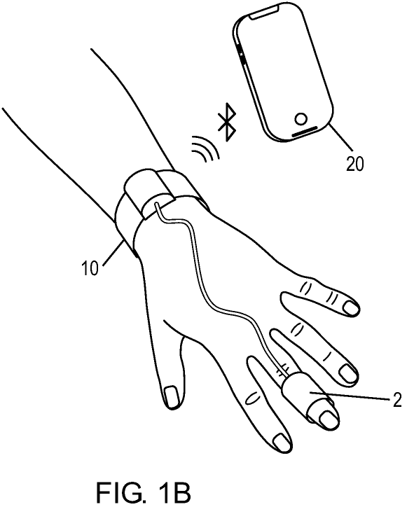

FIGS. 1A and 1B show example illustrations for implementing one or more embodiments of the device, system, and method for measuring vital signs including blood oxygen saturation, the system including a wired finger sensor device 1 or wired fingertip sensor device 2, wired wrist module device 10, and relay device 20.

As illustrated in FIG. 1A, the wired finger sensor device 1 may be ring-shaped to provide comfort to the wearer’s finger, encloses or wraps around the finger, and does not impede dexterity. This arrangement of the ring-shaped finger sensor provides a high quality stable signal for blood oxygen saturation (SpO₂) measurement, an indirect and noninvasive method of measuring oxygen saturation in blood at the finger. One of ordinary skill in the art, however, recognizes that the finger sensor device is not limited to the ring-shape. The finger sensor device 1 may include a pulse oximeter used to measure photoplethysmographic (PPG) signals to determine SpO₂. The measured PPG signal data may then be sent to the wired wrist module device 10 via a wire as depicted in FIG. 1A, which in turn may be wirelessly transmitted to the relay device 20 that calculates the SpO₂ using the received measured PPG signal data.

As illustrated in FIG. 1B, the wired fingertip sensor device 2 may be cylindrical to provide comfort to the wearer’s finger, encloses or wraps around the tip of a finger, and does not impede dexterity. This arrangement of the fingertip sensor device 2 provides a high quality stable signal for blood oxygen saturation (SpO₂) measurement, an indirect and noninvasive method of measuring oxygen saturation in blood at the finger. One of ordinary skill in the art, however, recognizes that the fingertip sensor device 2 is not limited to the cylindrical shape. The fingertip sensor device 2 may include a pulse oximeter used to measure photoplethysmographic (PPG) signals to determine SpO₂. The measured PPG signal data may then be sent to the wired wrist module device 10 via a wire as depicted in FIG. 1B, which in turn may be wirelessly transmitted by the wired wrist module device 10 to the relay device 20 that calculates the SpO₂ using the received measured PPG signal data.

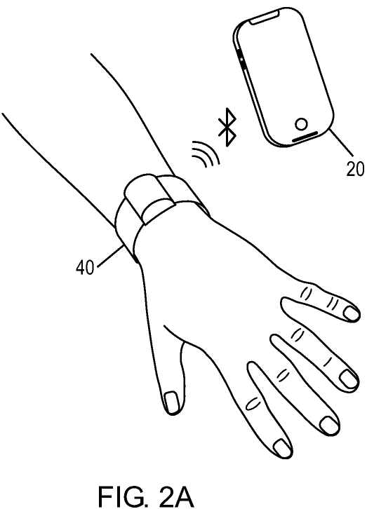

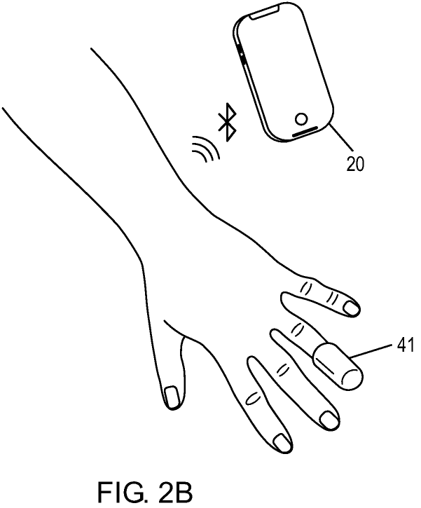

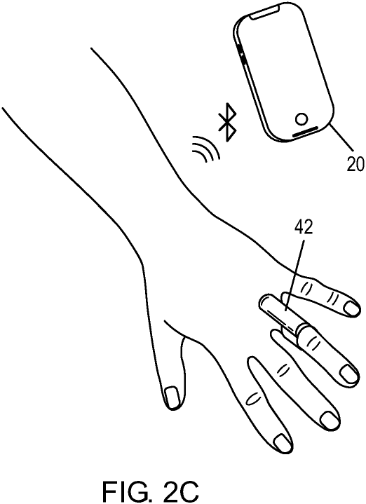

As illustrated in FIG. 2A, a wireless wrist sensor device 40 includes a band to provide comfort to the wearer’s wrist, encloses or wraps around the wrist, and does not impede dexterity. This arrangement of wireless wrist sensor device 40 provides a high quality stable signal for blood oxygen saturation (SpO₂) measurement at the wrist. The wireless wrist sensor device 40 may include a pulse oximeter used to measure photoplethysmographic (PPG) signals to determine SpO₂. The measured PPG signal data may then be sent wirelessly by the wireless wrist sensor device 40 to the relay device 20 to calculate the SpO₂ using the received measured PPG signal data.

As illustrated in FIG. 2B, a wireless fingertip sensor device 41 encloses or wraps around a user’s fingertip to provide comfort to the wearer’s finger and does not impede dexterity. This arrangement of the wireless fingertip sensor device 41 provides a high quality stable signal for blood oxygen saturation (SpO₂) measurement at the finger. The wireless fingertip sensor device 41 may include a pulse oximeter used to measure photo plethysmographic (PPG) signals to determine SpO₂. The measured PPG signal data may then be sent wirelessly by the wireless fingertip sensor device 41 to the relay device 20 to calculate the SpO₂ using the received measured PPG signal data.

As illustrated in FIG. 2C, a wireless ring sensor device 42 may be ring-shaped to provide comfort to the wearer’s finger and does not impede dexterity. This arrangement of the wireless ring sensor device 42 provides a high-quality stable signal for blood oxygen saturation (SpO2) measurement at the finger. The wireless ring sensor device 42 may include a pulse oximeter used to measure photo plethysmographic (PPG) signals to determine SpO2. The measured PPG signal data may then be sent wirelessly by the wireless ring sensor device 42 to the relay device 20 to calculate the SpO₂ using the received measured PPG signal data.

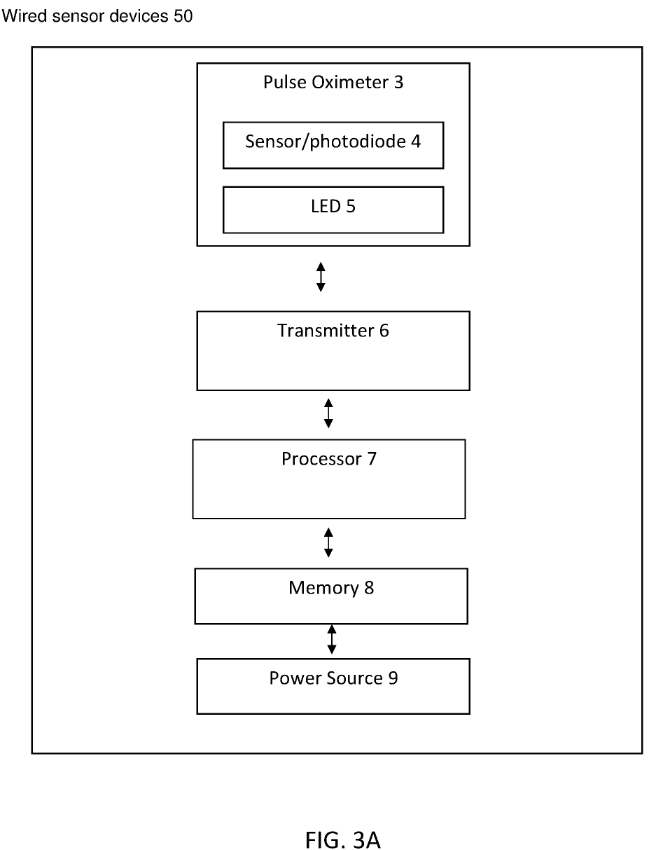

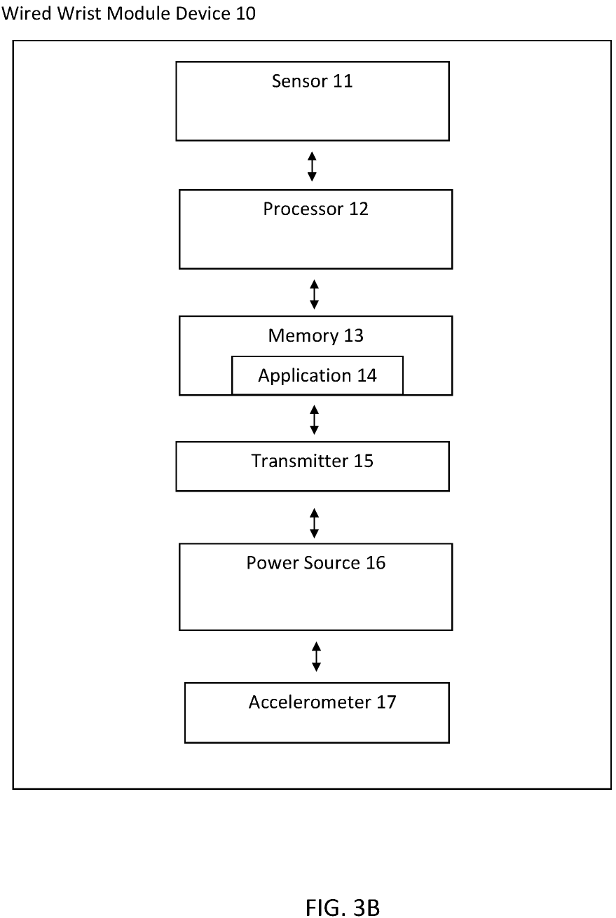

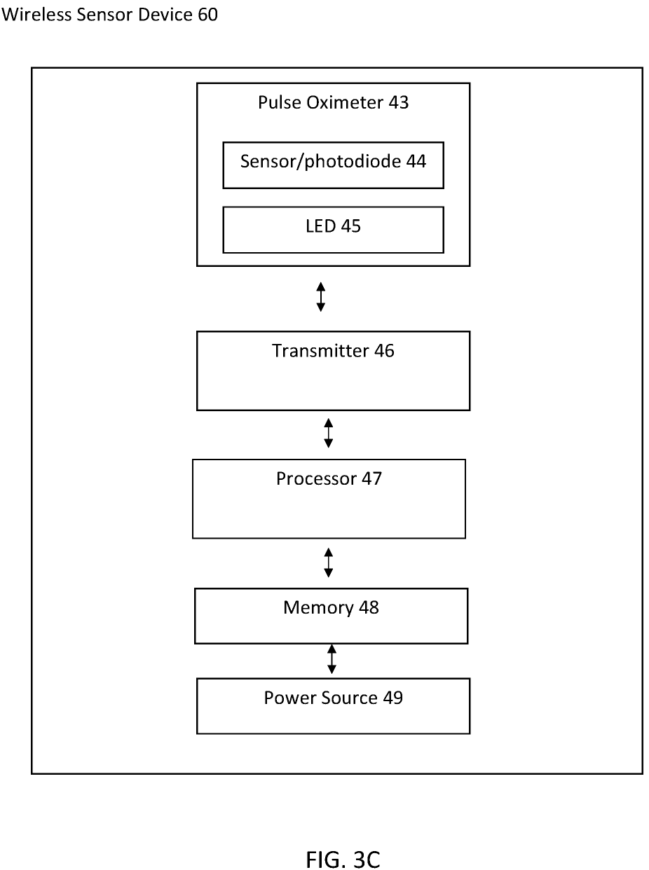

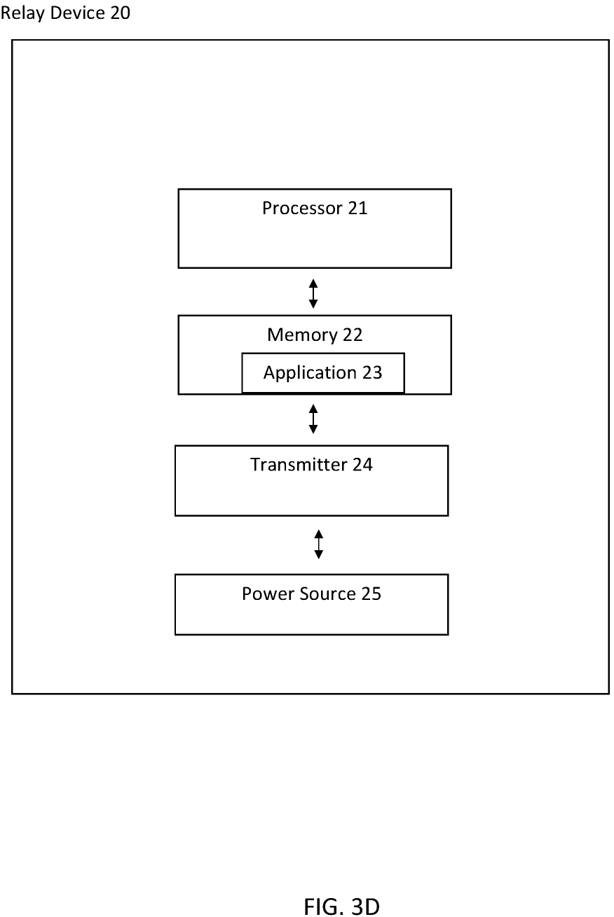

As illustrated in FIG. 3A, a block diagram of the hardware and software components of the wired finger sensor device 1 and wired fingertip sensor device 2 of FIGS. 1A and 1B, respectively, is described. For ease of description, the wired finger sensor device 1 and the wired fingertip sensor device 2 are collectively described as wired sensor devices 50.

The wired sensor devices 50 may include a pulse oximeter 3 which includes a sensor/photodiode 4 and at least one of an infrared light source, red light source, multiwavelength light source, or LED light source 5, a transmitter 6, coupled to the pulse oximeter 3, and a processor 7 coupled to a memory 8. The LED 5 may include one or more LEDs. The wired sensor devices 50 may be communicatively connected via transmitter 6 to the wired wrist module device 10 via wire or cable. The wired sensor devices 50, and/or wired wrist module device 10 may also be communicatively connected to the relay device 20, and to each other, via wireless (BLE) communication. The wired sensor devices 50 may be powered by the relay device 20 via a cabled connection. The wired sensor devices 50 may further include at least a seven-day portable power source 9 such as a rechargeable or non-rechargeable battery. The pulse oximeter 3 of the wired sensor devices 50 may noninvasively measures the oxygen saturation of a patient’s blood utilizing one of two methods of SpO₂ technology including transmission pulse oximetry and reflection pulse oximetry.

In an exemplary embodiment, transmissive pulse oximetry includes the photodiode 4 and the LED(s) 5 being placed on opposite sides of the human body part being measured (e.g., finger, wrist). Transmission pulse oximetry technology transmits red and infrared light from the LED(s) 5 through the body part to a photo detector (photodiode) 4. Oxygenated hemoglobin (O₂Hb) and deoxygenated hemoglobin (HHb) absorb red and infrared light differently. The body tissue absorbs some of the light, and the photodiode 4 collects the residual light that passes through the body.

In another exemplary embodiment, reflective pulse oximetry includes the photodiode 4 and the LED(s) 5 being on the same side of the human body part being measured (e.g., finger or wrist). The photodiode 4 collects the light reflected from various depths underneath the skin. The pulsatile arterial blood absorbs and modulates the incident light passing through the tissue and forms the photo plethysmographic (PPG) signal. The AC component of the PPG signals represents the light absorbed by the pulsatile arterial blood. This AC component is superimposed on a DC signal that captures the effects of light absorbed by other blood and tissue components (e.g., venous and capillary blood, bone, water, etc.). The ratio of the AC signal to the DC level is called the perfusion index (PI). The DC and AC components of the received PPG signals are different for different LED wavelengths. This is due to the different absorption characteristics of HbO₂, RHb, and other tissue components for different wavelengths. For example, to measure SpO₂, two LEDs with different wavelengths are utilized. In addition, these two wavelengths may be selected such that the molar absorption coefficients of HbO₂ and RHb are well separated. A red LED at, for example, 660 nm and an infrared LED at, for example, 880 nm are used in pulse oximetry. The pulse oximeter measures the PPG signals and the data is sent to the wired wrist module device 10 via a wire, and in turn the wired wrist module device 10 sends the PPG signal data to the relay device 20 to calculate the SpO₂. Alternatively, the pulse oximeter 3 measures the PPG signals and the data is sent directly via a wireless transmitter 6 to the relay device 20 to calculate the SpO₂.

As illustrated in FIG. 3B, a block diagram of the hardware and software components of the wired wrist module device 10 of FIGS. 1A and 1B is described. In an exemplary embodiment, the wired wrist module device 10 may be disposable, and may include electronics such as a sensor 11, a processor 12 coupled to a memory 13, an application 14 coupled to the memory 13, and a transmitter 15 coupled to the application. The wired wrist module device 10 may also provide continuous PPG signal measurement for SpO2 determination by being communicatively connected to the wired sensor devices 50 via a data wire or data cable. For example, the wired wrist module device 10 receives the PPG signals and data from the wired finger sensor device 1 or wired fingertip sensor device 2, and in turn the wired wrist module device 10 sends the PPG signals to the relay device 20. The wired wrist module device 10 may further include at least a seven-day portable power source 16 such as a battery which powers the wrist module device 10 and may also power the wired sensor devices 50. The battery may be rechargeable. Further, the wired wrist module device 10 may not include a screen or display to thereby reduce cost and size.

Further still, the wired wrist module device 10 may include an accelerometer 17 to detect motion artifacts. The accelerometer may include, but is not limited to, uni-axial accelerometers, bi-axial accelerometers, tri-axial accelerometers, gyroscopes, that would be within the spirit and scope of the present invention. The accelerometer of the wired wrist module device 10 may measure an analog accelerometer (ACC) signal of the user which is sent to the transmitter and in turn transmitted to the relay device 20 which subsequently utilizes the ACC signal to determine motion artifacts. The transmitter utilizes, for example, BLE wireless communication. One of ordinary skill in the art readily recognizes that the wired wrist module device 10 may utilize a variety of devices for the processor including but not limited to microprocessors, controllers, and microcontrollers and that would be within the spirit and scope of the present application. In addition, one of ordinary skill in the art readily recognizes that a variety of devices can be utilized for the memory, the application, and the transmitter and that would be within the spirit and scope of the present application.

As illustrated in FIG. 3C, a block diagram of the hardware and software components of the wireless wrist sensor device 40, wireless fingertip sensor device 41, and wireless ring sensor device 42 of FIGS. 2A, 2B, and 2C, respectively, is described. For ease of description, the wireless wrist sensor device 40, wireless fingertip sensor device 41, and wireless ring sensor device 42 are collectively described as wireless sensor devices 60. The wireless sensor devices 60 may include a pulse oximeter 43 which includes a sensor/photodiode 44 and at least one of an infrared light source, red light source, multiwavelength light source, or LED light source 45, a transmitter 46, coupled to the pulse oximeter 43, and a processor 47 coupled to a memory 48. LED light source 45 may further include one or more LEDs. The wireless sensor devices 60 may communicate wirelessly via transmitter 46 to the relay device 20 via wireless communication such as BLE communication. Wireless sensor devices 60 may be powered by at least a seven-day portable power source 49 such as a rechargeable or non-rechargeable battery. The pulse oximeter 43 of the wireless sensor devices 60 may noninvasively measures the oxygen saturation of a patient’s blood utilizing one of two methods of SpO2 technology including transmission pulse oximetry and reflection pulse oximetry.

As illustrated in FIG. 3D, a block diagram of the hardware and software components of the relay device 20 of FIGS. 1A and 1B is further described. The relay device 20 may include a processor 21, a memory 22 coupled to the processor 21, an application 23 stored in the memory 22, a power source 25 including battery power supply, and a transmitter 24 coupled to the application 23. The relay device 20 may be, for example, a tablet, smart phone, cell phone, or computer to carry out the tasks of computing, by using an algorithm, the underlying vital signs including PPG signal data for blood oxygen saturation Sp02, from the wrist module device 10, and wired finger sensor device 1 and/or wired fingertip sensor device 2. The relay device 20 may calculate the percentage of saturation of hemoglobin in arterial blood, SpO₂, using the measured light absorption changes caused by arterial blood flow pulsations.

In an exemplary embodiment, the relay device 20 receives the PPG signal data from the wired wrist module device 10, or wireless sensor devices 60, to calculate the SpO₂. The SpO₂ measurement is achieved by the following equation:

$${\text{SpO}}_{\text{2}} = a {R}^{2} + b R + c ,$$

where a, b, and c are calibration coefficients. Amplitudes of the absorbances are used to calculate the Red:IR Modulation Ratio (R), where R is determined by the following equation:

$$\text{R} = \left({\text{A}}_{\text{red,AC}} / {\text{A}}_{\text{red,DC}}\right) / \left({\text{A}}_{\text{IR,AC}} / {\text{A}}_{\text{IR,DC}}\right) \text{where A = absorbance} \text{.}$$

The AC component of the PPG signals represents the light absorbed by the pulsatile arterial blood. This AC component is superimposed on a DC signal that captures the effects of light absorbed by other blood and tissue components (e.g., venous and capillary blood, bone, water, etc.), and the ratio of the AC signal to the DC level is called the perfusion index (PI). In other words, R is a double-ratio of the pulsatile and non-pulsatile components of red-light absorption to IR light absorption. Regarding the calibration coefficients a, b, and c, a calibration process obtains calibration coefficients for better measurement accuracy by compensating for the deviations from the Beer-Lambert law and the non-idealities of the hardware. These coefficients may be obtained after collecting comprehensive data in a calibration lab, and using regression methods, fit a second (or first) order curve to the collected data. The fitted calibration curve is used to output the required calibration coefficients a, b, and c, calibration coefficients.

The computed underlying vital signs, including Sp02, are then displayed on the relay device 20 to report the results. Therefore, the use of the tablet, smart phone, or computer rather than including a display screen on the wired wrist module device 10, wired finger sensor device 1 and/or wired fingertip sensor device 2, or the wireless sensor devices 60, reduces cost and size, while providing a much higher computing processor power and far better display to report the result. Furthermore, the relay device 20 may be used to transmit the information to a central monitoring system that can further enhance the measurement, notify caregiver or interested parties and control the schedule and parameters of the measurement or the algorithm. By incorporating a small and inexpensive Bluetooth (or similar) module with the wired wrist module device 10, wired finger sensor device 1 and/or wired fingertip sensor device 2, or the wireless sensor devices 60, the raw signal is transmitted to the relay device 20. The relay device 20 then carries out all the necessary computations, displays the data on its screen and/or transmits the data to a central monitoring station.

With regard to the components and operations depicted in and described in accordance with FIGS. 1-3, any of the operations and sub-operations may be implemented as non-transitory computer-readable instructions stored on a computer-readable medium. The computer-readable instructions may, for example, be executed by the one or more processors of the relay device 20, wrist module device 10, wired finger sensor device 1 and/or wired fingertip sensor device 2, or the wireless sensor devices 60, as referenced herein, having a network element and/or any other device corresponding thereto, particularly as applicable to the applications and/or programs described above.

From the foregoing, it will be appreciated that various embodiments of the present disclosure have been described herein for purposes of illustration, and that various modifications may be made without departing from the scope and spirit of the present disclosure. Further, one of ordinary skill in the art would understand that the various embodiments may be combined or separated, and is within the possession of, consideration of, and contemplation of the inventors to combine various embodiments. Accordingly, the various embodiments disclosed herein are not intended to be limiting, with the true scope and spirit being indicated by the following claims.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Furthermore, the present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and even apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

There is little distinction left between hardware and software implementations of aspects of the above described systems; the use of hardware or software is generally (but not always, in that in certain contexts the choice between hardware and software can become significant) a design choice representing cost vs. efficiency tradeoffs. There are various vehicles by which processes and/or systems and/or other technologies described herein may be implemented, e.g., hardware, software, and/or firmware, and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes for determining blood oxygen saturation via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium, a hard disk drive, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate the devices and/or processes described herein into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “ a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “ a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A system to determine blood oxygen saturation (SpO₂), comprising:
a finger sensor device including a sensor and a transmitter coupled to the sensor;
a wrist module device communicatively connected to the finger sensor device via a cable; and
a relay device communicatively connected to the wrist module device;
wherein the sensor of the finger sensor device noninvasively measures changes of light absorption in oxygenated or deoxygenated blood as signal data and sends the signal data to the wrist module via the cable,
wherein the wrist module receives the signal data and wirelessly sends the signal data to the relay device,
wherein the relay device calculates a percentage of saturation of hemoglobin in the blood (Sp02) using the signal data, and
wherein the relay device displays the calculated percentage of saturation of hemoglobin in the blood.

2. The system of claim 1, wherein the relay device calculates the Sp02 via a calibrated algorithm using a double-ratio of the pulsatile and non-pulsatile components of red-light absorption to IR light absorption.

3. The system of claim 1, wherein the relay device is communicatively connected to the wrist module device via Bluetooth (BLE) wireless communication.

4. The system of claim 1, wherein the finger sensor device is ring-shaped.

5. The system of claim 1, wherein the finger sensor device includes a pulse oximeter.

6. The system of claim 5, wherein the pulse oximeter includes multiwavelength light sources, and photo detectors.

7. The system of claim 1, wherein the wrist module includes a power source that powers the wrist module and finger sensor device.

8. The system of claim 1, wherein the wrist module does not include a display screen.

9. The system of claim 1, wherein the finger sensor device does not include a display screen.

10. The system of claim 1, wherein the wrist module includes an accelerometer to detect motion artifacts.

11. The system of claim 10, wherein the accelerometer includes at least one of: uniaxial accelerometers, bi-axial accelerometers, tri-axial accelerometers, or gyroscopes.

12. The system of claim 10, wherein the wrist module includes a transmitter.

13. The system of claim 12,
wherein the accelerometer measures an analog accelerometer (ACC) signal of the patient and sends the ACC signal to the transmitter, and
wherein the transmitter transmits the ACC signal to the relay device which subsequently utilizes the ACC signal to determine the motion artifacts.

14. The system of claim 1, wherein the relay device includes at least one of a tablet, smart phone, or computer to calculate, by using an algorithm, the percentage of saturation of hemoglobin by using a received PPG signal data from the wrist module device.

15. The system of claim 14, wherein the relay device displays the calculated percentage of saturation of hemoglobin on a screen of the tablet, smart phone, or computer to report the results.

16. A method to determine blood oxygen saturation (SpO₂), comprising:
noninvasively measuring, by a finger sensor device, changes of light absorption in oxygenated or deoxygenated blood as signal data;
sending, by the finger sensor device, the signal data to a wrist module via a cable;
wirelessly sending, by the wrist module device, the signal data to a relay device;
calculating, by the relay device, a percentage of saturation of hemoglobin in the blood (Sp02) using the signal data; and
displaying the Sp02 on a display of the relay device.

17. The method of claim 16, wherein the calculating the Sp02 includes the relay device calculating the Sp02 via a calibrated algorithm using a double-ratio of the pulsatile and non-pulsatile components of red-light absorption to IR light absorption.

18. A biosensor device to determine blood oxygen saturation (SpO₂), comprising:
a finger sensor coupled to a transmitter; and
a wrist module communicatively connected to the finger sensor via a cable;
wherein the finger sensor noninvasively measures changes of light absorption in oxygenated or deoxygenated blood as data and sends the data to the wrist module via the cable,
wherein the wrist module receives the data and wirelessly sends the data to a relay device,
wherein the relay device calculates a percentage of saturation of hemoglobin in the blood (Sp02) using the data, and
wherein the relay device displays the calculated percentage of saturation of hemoglobin in the blood.

19. The biosensor device of claim 18, wherein the wrist module includes a power source that powers the wrist module and finger sensor.

20. The biosensor device of claim 18, wherein the relay device calculates the Sp02 via a calibrated algorithm using a double-ratio of the pulsatile and non-pulsatile components of red-light absorption to IR light absorption.
